(19) United States
(12) Patent Application Publication
(10) Pub. No.: US 2015/0125402 A1
(43) Pub. Date: May 7, 2015

(54) ANTIMICROBIAL AGENTS

(71) Applicant: BEDOUKIAN RESEARCH, INC., Danbury, CT (US)

(72) Inventor: Robert H. Bedoukian, West Redding, CT (US)

(21) Appl. No.: 14/391,313

(22) PCT Filed: Apr. 30, 2013
(86) PCT No.: PCT/US13/00118

§ 371 Date: Oct. 8, 2014

Related U.S. Application Data
(60) Provisional application No. 61/741,364, filed on Jul. 18, 2012.

(51) Int. Cl. A01N 35/06; A23L 3/34; A01N 31/06; A23G 4/06; C07C 49/603; C07C 35/18

(52) U.S. Cl. CPC A01N 35/06; C07C 49/603; C07C 35/18; A01N 31/06; A23G 4/06; A23L 3/34; A23V 2002/00
USPC 424/48; 514/690; 424/65; 424/49; 514/729; 426/3

(57) ABSTRACT

Compounds are used in compositions as antimicrobial agents against microbes, such as for example, Staphylococcus aureus, Staphylococcus epidermidis, Escherichia coli, Pseudomonas aeruginosa, Candida albicans, and Aspergillus brasiliensis. Antimicrobial action is obtained by contact of a microorganism with at least one of the compounds of the structure (I); wherein R is a branched or straight chain, saturated or unsaturated hydrocarbyl group with zero or one double bond and a total of from 1 to 11 carbon atoms; R¹ is —OH, =0 or —OC(0)CH₃ and wherein the compounds of structure (I) contain a total of from 9 to 18 carbon atoms in the compounds.

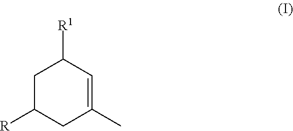

### FIELD OF THE INVENTION

This invention relates to the use of compounds as agents that deter microbial growth in a variety of microorganisms. The compounds of this invention are an effective antimicrobial control agent against microorganisms such as, for example, Staphylococcus aureus, Staphylococcus epidermidis, Escherichia coli, Pseudomonas aeruginosa, Candida albicans, and Aspergillus brasiliensis.

### BACKGROUND TO THE INVENTION

Microorganisms (aka microbes), including but not limited to, Staphylococcus aureus, Staphylococcus epidermidis, Escherichia coli, Pseudomonas aeruginosa, Candida albicans, and Aspergillus brasiliensis, are recognized to be causative agents in a variety of infectious diseases.

Antimicrobial agents have been utilized to either kill microbes (microbiocidal agents) or prevent the growth of microbes (microbiostatic agents). Both modes of action are important because both decrease the number of disease causing microorganisms.

There are a number of current means of control growth of microorganisms. Some fragrance ingredients, either alone or combined with other bacteriostatic agents, have been patented to control microbes, such as for example as disclosed in U.S. Pat. Nos. 5,306,707; 5,420,104; 5,965,518 and 6,241,979 B1. However, such compositions have not been found to be very effective antimicrobial compositions. Additionally, the article by J. A. Morris et al., Antimicrobial Activity of Aroma Compounds and Essential Oils, Journal of the American Chemical Society, Vol. 56, 595-603, May 1979, concludes, from their testing on 521 fragrance ingredients, that “Thus, the creation of a practical fragrance with significant antimicrobial activity appears highly unlikely”.

There is therefore a need for compounds that have sufficient antimicrobial activity so as to be suitable for use in formulations to provide antimicrobial activity and also present fragrance and flavoring properties to the formulations.

### SUMMARY OF THE INVENTION

In accordance with this invention, an antimicrobial effect against microbes is obtained by contact of a microorganism with an antimicrobial effective amount of at least one of the compounds of the structure (I)

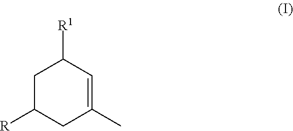

wherein R is selected from a branched or straight chain, saturated or unsaturated hydrocarbyl group with zero or one double bond and contains from 1 to 11 carbon atoms; and R¹ is selected from the group consisting of —OH, ═O and —OC(O)CH₃ and wherein the compounds of structure (I) contain a total of from 9 to 18 carbon atoms in the compounds. The invention also includes optical isomers, diastereomers and enantiomers of the named structures. Thus, at all stereocenters where stereochemistry is not explicitly defined, all possible epimers are envisioned.

The compounds of structure (I) are effective antimicrobial agents against a wide variety of microorganisms, including, but not limited to, Staphylococcus aureus, Staphylococcus epidermidis, Escherichia coli, Pseudomonas aeruginosa, Candida albicans, and Aspergillus brasiliensis. Additionally the antimicrobial agents of this invention may possess fragrance and flavoring properties. Furthermore, the antimicrobial compounds of this invention are known to possess the ability to control or repel various insects, mosquitoes and bed bugs as disclosed in Applicant's co-pending U.S. Provisional Patent Application Nos. 61/687,917; 61/687,918; and 61/687,919 thereby making them extremely useful in a variety of formulations.

### DETAILED DESCRIPTION OF THE INVENTION

Antimicrobial effects against microbes, including, but not limited to, Staphylococcus aureus, Staphylococcus epidermidis, Escherichia coli, Pseudomonas aeruginosa, Candida albicans, and Aspergillus brasiliensis, are obtained by contact of a microorganism with an antimicrobial effective amount of at least one of the compounds of the structure (I)

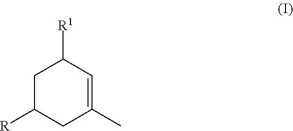

wherein R is a branched or straight chain, saturated or unsaturated hydrocarbyl group with zero or one double bond and containing from 1 to 11 carbon atoms; R¹ is selected from —OH, ═O and —OC(O)CH₃, and wherein the compounds of structure (I) contain a total of from 9 to 18 carbon atoms in the compounds. The invention also includes optical isomers, diastereomers and enantiomers of the named structures. Thus, at all stereocenters where stereochemistry is not explicitly defined, all possible epimers are envisioned.

The active compounds of structure (I) may be employed in any suitable formulation, such as, but not limited to, liquid cleansers, spray cleansers, wipes, soaps, deodorants, antiperspirants, body sprays and oral care products such as mouth drops, chewing gums, toothpastes, mouthwashes, mouth sprays and candies. The amount of the antimicrobial effective amount of at least one compound of structure (1) in such formulations will vary depending upon the specific formulation and also may vary according to the microorganism that is desired to be combatted. Generally the amount of at least one compound of structure (1) in the formulation will be an amount of from about 5 ppm to about 5000 ppm, preferably from about 10 ppm to about 500 ppm.

Additionally, the active compounds of structure (I) may be employed in combination with other recognized antimicrobial agents. Such other antimicrobial agents include, but are not limited to, triclosan, 2,2-methylene bis (3,4,6 trichlorophenol), 2,4,4′-trichlorocarbanilide, 3,4,4′-trichlorocarbanilide, 2,5,4′-tribromosalicylanilide, 3-trimethylfluro-4,4′-dichlorocarbanilide, dichlorophenol, trichlorosalycilanilide, and tetrachlorosalycilanilide. Other possible antibacterial agents include, but are not limited to, those listed in McCutcheon's Functional Materials (1995 North American Edition, pages 2-10), which disclosure is incorporated herein by this reference thereto.

Representative examples of compounds of structure (I) include, but are not limited to,

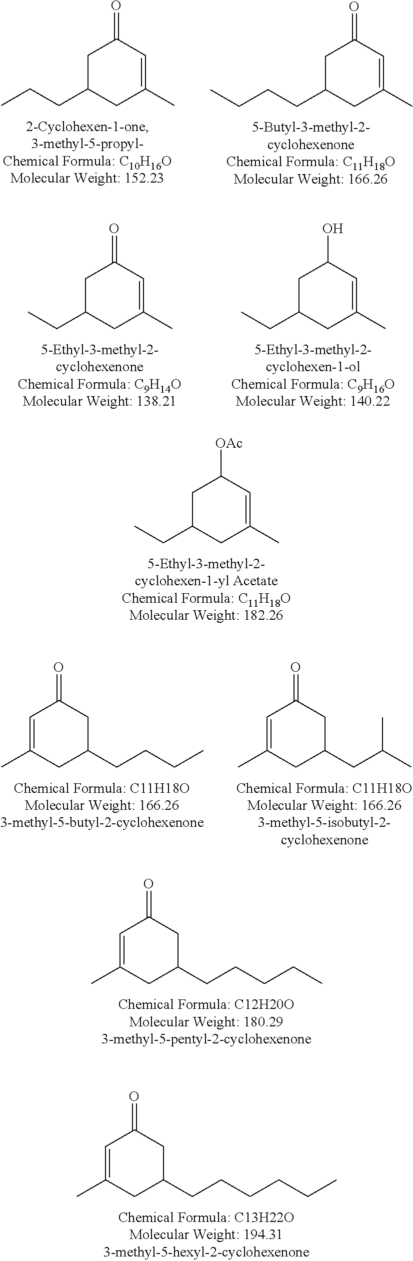

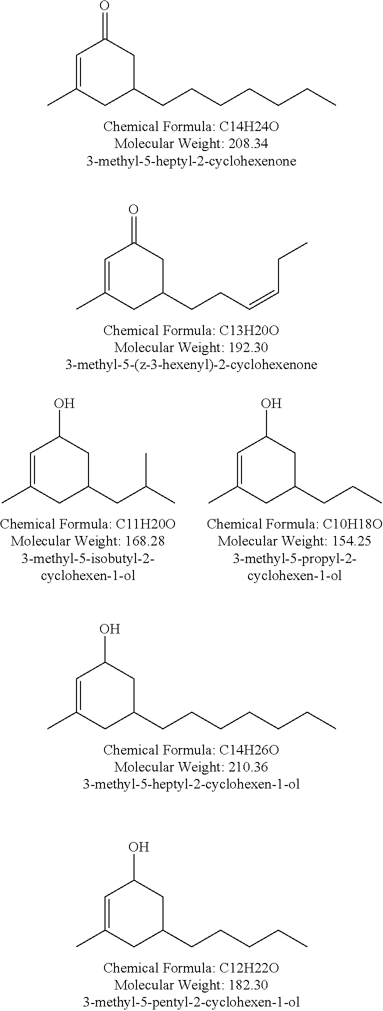

Preferred compounds of structure (I) for use as antimicrobial agents include 3-methyl-5-propyl-2-cyclohexenone, 3-methyl-5-butyl-2-cyclohexenone, 3-methyl-5-butyl-2-cyclohexenol and 3-methyl-5-heptyl-2-cyclohexenone.

To test the compounds as antimicrobial agents, the compounds were screened for activity to inhibit bacteria, yeast and mold. The Minimum Inhibition Concentration (MIC) is determined as the lowest concentration activity to inhibit the microorganism. Therefore smaller number means more active. From the data table below, each of the materials showed some activity for the microorganisms tested. 3-methyl-5-butyl-2-cyclohexenone was slightly more broader spectrum while 3-methyl-5-heptyl-2-cyclohexenone was more active against Gram positive bacteria. 3-methyl-5-butyl-2-cyclohexenol was more active against Gram positive bacteria and fungi than the Gram negative bacteria tested.

TABLE 1

| | | | Microorganism | | | | | |
|---|---|---|---|---|---|---|---|---|
| | | | Staphylococcus | Staphylococcus | Escherichia | Pseudomonas | Candida | Aspergillus |
| | | | aureus | epidermidis | coli | aeruginosa | albicans | brasiliensis |
| Strain | ATTC | | 6538 | 12228 | 10536 | 15442 | 10231 | 16404 |
| 3-Methyl-5- | MIC | ppm | 500 | 1000 | 500 | >1000 | 500 | 250 |
| Butyl-2- | | | | | | | | |
| Cyclohexenone | | | | | | | | |
| 3-Methyl-5- | MIC | ppm | 125 | 125 | >1000 | >1000 | 125 | 250 |
| Butyl-2- | | | | | | | | |
| Cyclohexenol | | | | | | | | |
| 3-Methyl-5- | MIC | ppm | 1000 | >1000 | 1000 | >1000 | 1000 | 500 |
| Propyl-2- | | | | | | | | |
| Cyclohexenone | | | | | | | | |
| 3-Methyl-5- | MIC | ppm | 15.6 | 31.25 | >1000 | >1000 | >1000 | >1000 |
| Heptyl-2- | | | | | | | | |
| Cyclohexenone | | | | | | | | |

In a second assay, compounds of this invention were screened for activity to kill bacteria in solution over a short period of time. Bactericidal activity is expressed as Activity % Reduction. All samples were tested in water at ambient temperature and at a concentration of 2000 ppm. From the data table below the materials showed activity as tested. 3-methyl-5-butyl-2-cyclohexenone was slightly more active than 3-methyl-5-propyl-2-cyclohexenone or 3-methyl-5-butyl-2-cyclohexenol against only E. coli and not S. aureus at the concentrations and time intervals tested.

TABLE 2

| | Staphylo- | Staphylo- | | |
|---|---|---|---|---|
| | coccus | coccus | Escherichia | Escherichia |
| | aureus | aureus | coli | coli |
| Test Compound | 5 minutes | 1 hour | 5 minutes | 1 hour |
| 3-Methyl-5-Butyl- | 33.90% | 33.20% | 83.78% | 99.96% |
| 2-Cyclohexenone | | | | |
| 3-Methyl-5-Butyl- | 28.40% | 39.00% | 34.70% | 88.52% |
| 2-Cyclohexenol | | | | |
| 3-Methyl-5- | 29.20% | 33.90% | 69.45% | 98.96% |
| Propyl-2- | | | | |
| Cyclohexenone | | | | |
| 3-Methyl-5- | 37.60% | 24.10% | 13.90% | 25.00% |
| Heptyl-2- | | | | |
| Cyclohexenone | | | | |

I claim:

1. A method for deterring microbial growth in a microorganism, the method comprising contacting a microorganism with an antimicrobial effective amount of from about 5 ppm to about 5000 ppm of at least one of compound of the structure (I) 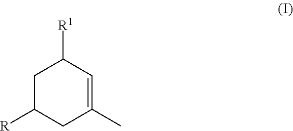
wherein R is a branched or straight chain, saturated or unsaturated hydrocarbyl group with zero or one double bond and contain a total of from 1 to 11 carbon atoms; R¹ is selected from the group consisting of —OH, ═O and —OC(O)CH₃ and wherein the compounds of structure (I) contain a total of from 9 to 18 carbon atoms in the compounds.

2. The method according to claim 1 wherein the microorganism is selected from the group consisting of Staphylococcus aureus, Staphylococcus epidermidis, Escherichia coli, Pseudomonas aeruginosa, Candida albicans, and Aspergillus brasiliensis.

3. The method according to claim 1 wherein the compound of structure (I) is selected from the group consisting of:

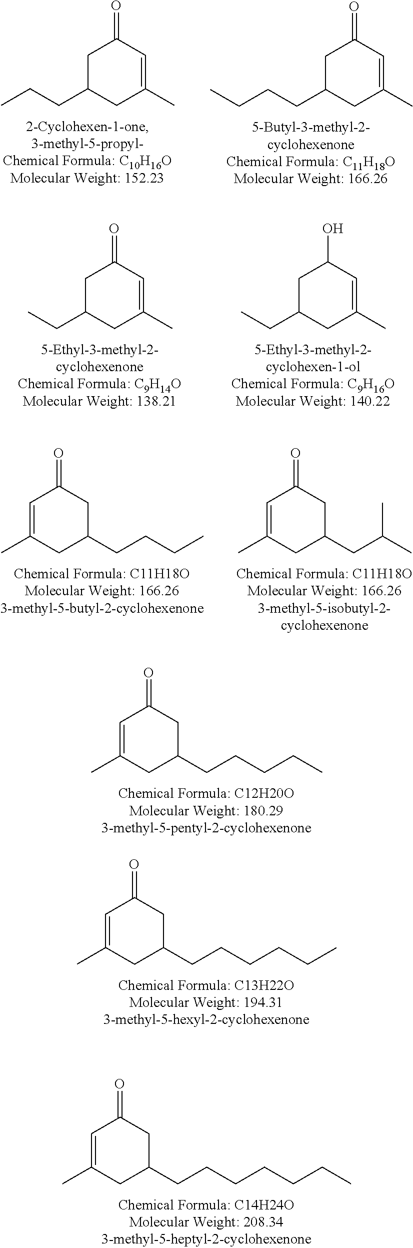

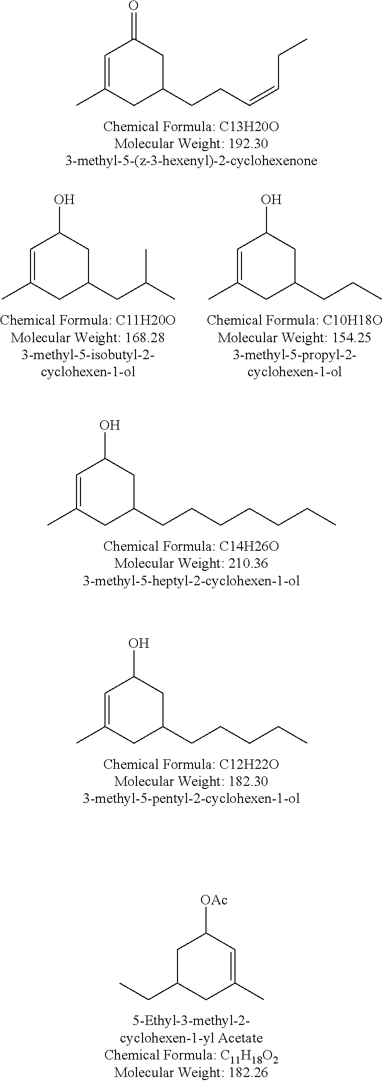

4. The method according to claim 1 wherein at least one compound of structure (I) is utilized in a formulation selected from the group consisting of liquid cleansers, spray cleansers, wipes, soaps, deodorants, antiperspirants, body sprays, mouth drops, chewing gums, toothpastes, mouthwashes, mouth sprays and candies.

5. The method of claim 1 wherein the compound of structure (I) is selected from the group consisting of: 3-methyl-5-propyl-2-cyclohexenone, 3-methyl-5-butyl-2-cyclohexenone, 3-methyl-5-butyl-2-cyclohexenol and 3-methyl-5-heptyl-2-cyclohexenone.

6. The method of claim 1 wherein the at least one compound of structure (I) is contained in a formulation in an amount of from about 10 ppm to about 500 ppm.

7. A composition for deterring microbial growth in a microorganism by contacting a microorganism with an antimicrobial effective amount of the composition wherein the composition contains from about 5 ppm to about 5000 ppm of at least one of compound of the structure (I) 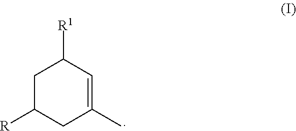
wherein R is a branched or straight chain, saturated or unsaturated hydrocarbyl group with zero or one double bond and contain a total of from 1 to 11 carbon atoms; R¹ is selected from the group consisting of —OH, ═O and —OC(O)CH₃ and wherein the compounds of structure (I) contain a total of from 9 to 18 carbon atoms in the compounds.

8. The composition according to claim 7 wherein the compound of structure (I) is selected from the group consisting of:

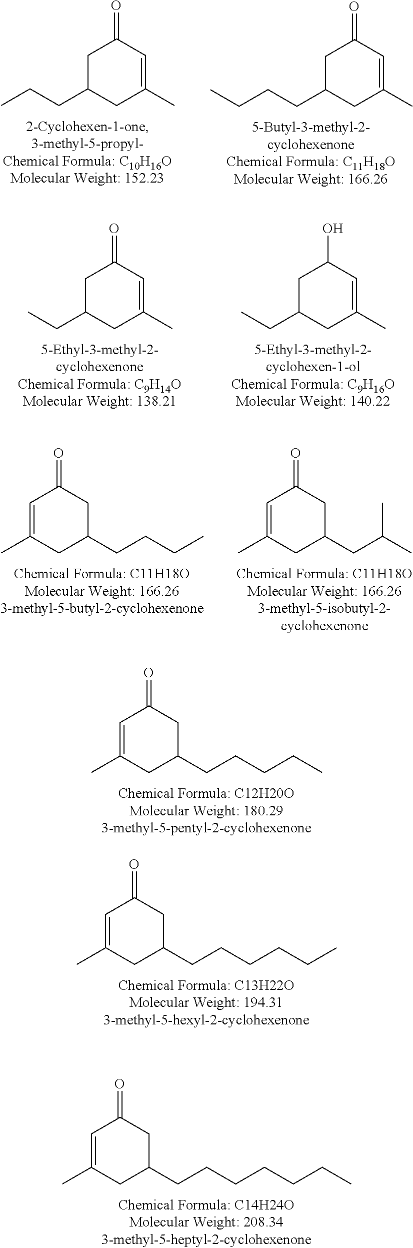

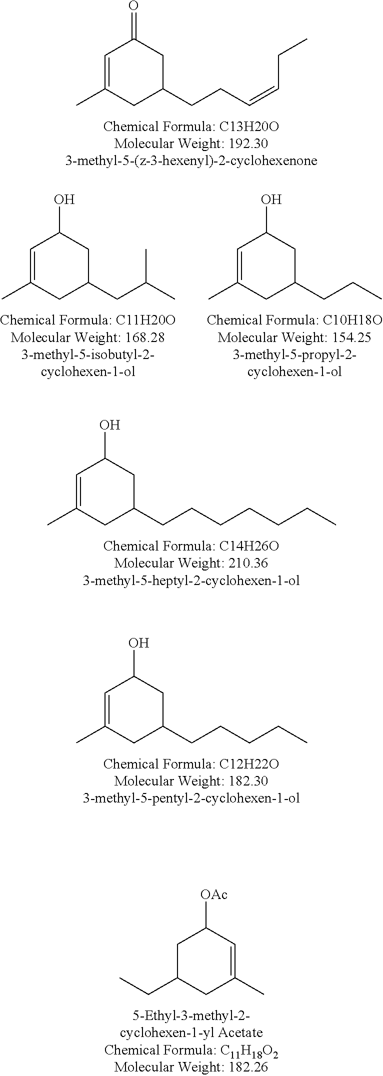

9. The composition according to claim 7 wherein the composition is a formulation selected from the group consisting of liquid cleansers, spray cleansers, wipes, soaps, deodorants, antiperspirants, body sprays, and oral care products such as, mouth drops, chewing gums, toothpastes, mouthwashes, mouth sprays and candies.

10. The composition of claim 7 wherein the compound of structure (I) is selected from the group consisting of: 3-methyl-5-propyl-2-cyclohexenone, 3-methyl-5-butyl-2-cyclohexenone, 3-methyl-5-butyl-2-cyclohexenol and 3-methyl-5-heptyl-2-cyclohexenone.

11. The composition of claim 7 wherein the at least one compound of structure (I) is contained in a formulation in an amount of from about 10 ppm to about 500 ppm.

12. The composition of claim 7 wherein the composition contains at least one additional recognized antimicrobial agent.

13. The composition of claim 12 wherein the at least one additional recognized antimicrobial agent is selected from the group consisting of triclosan, 2,2-methylene bis (3,4,6 trichlorophenol), 2,4,4′-trichlorocarbanilide, 3,4,4′-trichlorocarbanilide, 2,5,4′-tribromosalicylanilide, 3-trimethylfluro-4,4′-dichlorocarbanilide, dichlorophenol, trichlorosalycilanilide, and tetrachlorosalycilanilide.
